(19) United States
(12) Patent Application Publication
(10) Pub. No.: US 2002/0173756 A1
(43) Pub. Date: Nov. 21, 2002

(54) Ophthalmic injector

(76) Inventor: Terence Arnold Waldock, Meppershall (GB)

Correspondence Address: Woodcock washburn, One Liberty Place - 46th Floor, Philadelphia, PA, 19103, US

(21) Appl. No.: 10/019,981

(22) PCT Filed: May 8, 2001
(86) PCT No.: PCT/GB01/02023

(30) Foreign Application Priority Data
May 6, 2000 (GB) 0010871.2

(51) Int. Cl. A61M 35/00

(52) U.S. Cl. 604/294

(57) ABSTRACT

An ophthalmic injector for the insertion of segments of material into the eye comprises a barrel (11), a plunger (24) displaceable within the barrel, a nose (14) and a nose tip (16). A rotatable carousel-type holder (22) for the segments is mounted in the nose for stepwise indexing movement. The segments are pushed in turn from the holder by a centre rod (30) connected to the plunger, through the nose and along a guide channel in the nose tip. The nose (14) and the nose tip (16) are pivotable through 90° relative to the barrel. Alternatively, the segment holder can be a linearly movable holder, indexable transversely across and through the barrel. The holder can carry nine segments. The injector can be used for the insertion of segments used in the treatment of hyperopia.

### FIELD OF THE INVENTION

This invention relates generally to ophthalmic surgical instruments, and particularly to ophthalmic injectors by means of which segments of material can be inserted into incisions in the eye. It is more particularly concerned with instruments for the surgical treatment of conditions such as hyperopia and presbyopia where segments of material are inserted into the eye-for corrective treatment.

Hyperopia is a condition in which visual images come to a focus behind the retina of the eye because of defects in the refractive media of the eye or because of abnormal shortness of the eyeball. A new surgical technique for the treatment of this condition involves the insertion of a plurality of segments of transparent material, e.g. PMMA, into the cornea, to flatten the cornea and to change the refractive parameters of the eye. The technique involves making incisions in the cornea on a circle towards the outside of the cornea. The incisions are generally about 400 microns deep, i.e. about two-thirds of the thickness of the cornea, and six to eight incisions are normally made, equispaced around the cornea. Specially shaped segments of transparent material are then pushed into the incisions, from the outside of the cornea towards the optical zone. These segmental inserts have the effect of flattening the cornea and changing the refractive parameters in such a way as to bring the visual images to a focus on the retina.

### SUMMARY OF THE INVENTION

It is an object of the present invention to provide a surgical instrument which will facilitate the introduction of segmental inserts into the eye by the surgeon.

It is a further object of the invention to provide an ophthalmic injector for the treatment of for example hyperopia which can be used with segmental inserts of different dimensions without the need to adjust the injector.

It is yet a further object of the invention to provide an ophthalmic injector for the treatment of for example hyperopia which enables the surgeon to perform the treatment more easily and more accurately than using forceps, which is what is currently used.

FIG. 1 of the accompanying drawings illustrates a typical segmental insert, indicated generally at 10, as used in the treatment of hyperopia. FIGS. 1a to 1c show the insert in plan view, side view and cross-section respectively. The numerical ranges shown in the drawing in respect of length, width and thickness illustrate the range of dimensions which is typical for these inserts. The dimensions are given in inches. The corresponding metric values are as follows:

| | |
|---|---|
| Length: | 1.50-2.16 mm |
| Width: | 0.48-0.79 mm |
| Thickness: | 0.28-0.51 mm |

The surgeon will choose segments of appropriate dimensions to meet the needs of individual patients. For any given eye, each of the segments which is inserted will be of the same dimensions.

In accordance with the invention there is provided an ophthalmic injector comprising a barrel, a plunger displaceable longitudinally within the barrel, a nose tip forwardly of the barrel, and a segment holder arranged to hold a plurality of segments to be inserted into incisions in the eye, the segment holder being displaceable stepwise and being positioned so that the segments are engageable in turn by the plunger or an extension thereof to push them from the holder through the nose tip.

The segment holder may be a rotatable carousel-type holder with segments arranged on the locus of a circle, or may alternatively be a linear holder which can be indexed through the barrel.

Preferably, the segments in the holder are retained under spring bias until pushed from the holder, to enable different sizes of segments to be accommodated in the same holder.

The barrel of the injector may be a solid element or may alternatively be of the type which can be broken open at a nose portion, in the manner of a shotgun, for the insertion of the segment holder.

### BRIEF DESCRIPTION OF THE DRAWINGS

In order that the invention may be more fully understood, two presently preferred embodiments of hyperopia injector will now be described by way of example and with reference to the accompanying drawings. In the drawings:

FIG. 2 is a top plan view of a first embodiment of injector in accordance with the invention, with the plunger depressed;

FIG. 3 is a side view of the injector of FIG. 2, with the plunger retracted;

FIG. 4 is a top plan view of the nose tip of the injector of FIGS. 2 and 3;

FIG. 5 is a side view of the nose tip of FIG. 4;

FIG. 6 is an end view of the nose tip of FIGS. 4 and 5;

FIG. 7 is a side view of the nose of the injector of the first embodiment;

FIG. 8 is an end view of the nose of FIG. 7, viewed from the left-hand side of FIG. 7;

FIG. 9 is an end view of the nose of FIG. 7, viewed from the right-hand side of FIG. 7;

FIG. 10 is an end view of the segment holder of the injector of FIGS. 2 and 3;

FIG. 11 Is a vertical sectional view through the centre of the segment holder shown in FIG. 10;

FIG. 12 is an end view of a segment holder spring of the injector of FIGS. 2 and 3;

FIG. 13 is a sectional view through the segment holder spring of FIG. 12;

FIG. 14 is a side view of the centre rod of the injector of FIGS. 2 and 3;

FIG. 15 is a view, on an enlarged scale, of the tip of the centre rod of FIG. 14;

FIG. 16 is a plan view of a loading plate for use with the injector of FIGS. 2 and 3;

FIG. 17 is a side view of the loading plate of FIG. 16.

FIG. 18 is a side view of a second embodiment of injector in accordance with the invention, with the plunger depressed;

FIG. 19 is an underneath plan view of the injector of FIG. 18, with the plunger retracted;

FIG. 20 shows the segment holder case used in the injector of FIGS. 18 and 19;

FIG. 21 is a cross-sectional view through the segment older case of FIG. 20;

FIG. 22 shows the spring used with the segment holder case of FIG. 20; and,

FIG. 23 is a cross-sectional view through the spring shown in FIG. 22.

### DESCRIPTION OF THE PREFERRED EMBODIMENTS

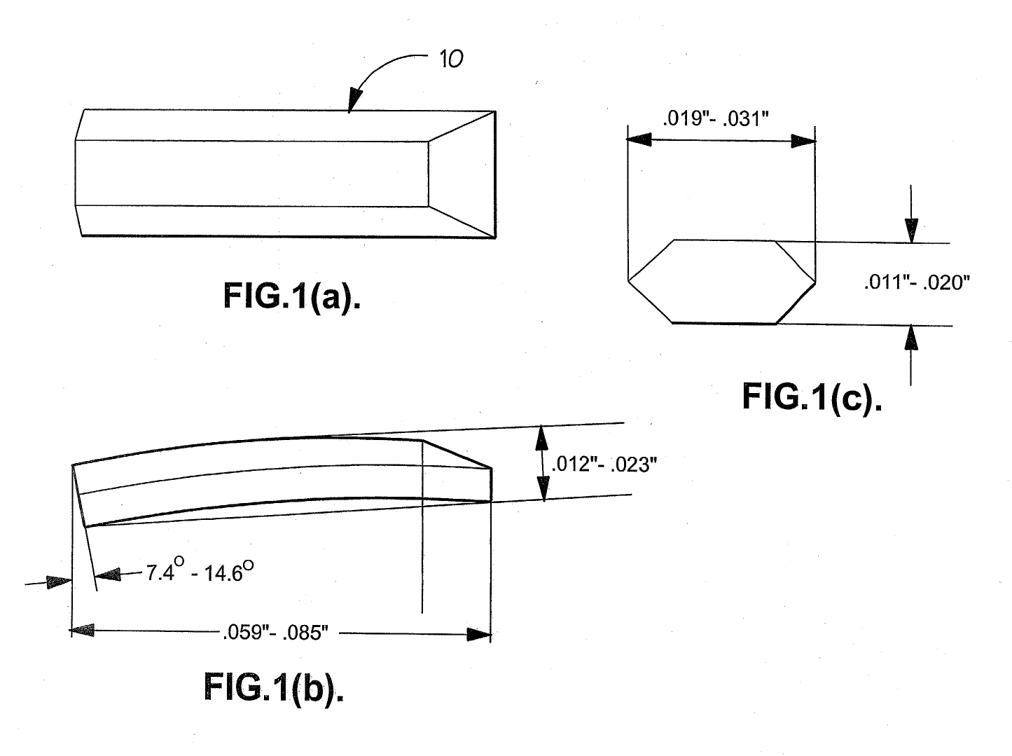

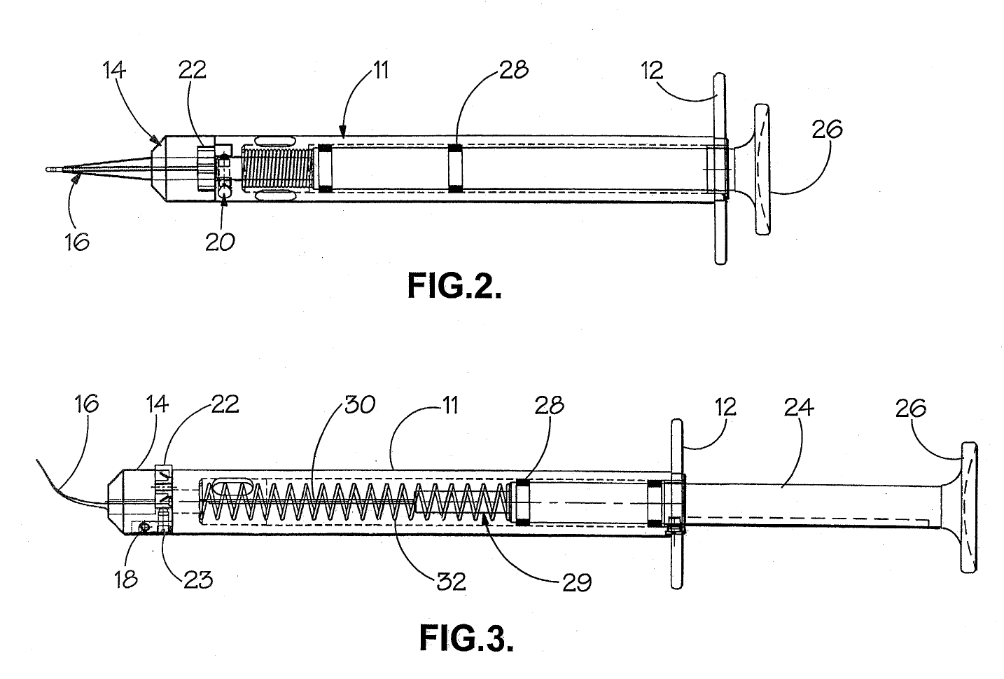

Referring first to FIGS. 2 and 3, there is shown a first embodiment of segment injector which is of the broken barrel type, i.e. the front end portion can be broken open relative to the main body of the instrument to facilitate loading of the segments to be inserted. The injector comprises a main body 11 which has a flange 12 at the rearward end. A nose 14 having a nose tip 16 is connected to the injector body 11 for pivotal movement about a pivot pin 18. Thus, the nose 14 and nose tip 16 can be pivoted down through approximately 90° relative to the injector body. When closed, the nose 14 is held locked in place by a locking spring, screw and domed pin, indicated generally at 20. The nose 14 houses a segment holder 22 which will be described in more detail hereinafter. As shown most clearly in FIG. 3, the segment holder projects proud of the main body of the injector so that it can be manually rotated. The segment holder is held in place in a manner which enables it to be indexed by the provision of a locking spring, screw and domed pin, indicated generally at 23.

Within the injector body 11 there is housed a plunger 24 which has a thumb pad 26 at its outer end and which carries a pair of friction pads 28 spaced along its length for engagement with the inside of the injector body 11. The forward end 29 of the plunger 24 is of reduced diameter and is counterbored to receive the rearward end of a centre rod 30 to which it is pinned. The purpose of the centre rod 30 is to push segments from the segment holder 22 out through the nose tip, as will be described in more detail hereinafter. Within the body 11 there is also provided a helical spring 32 which is seated at the forward end against an abutment surface within the injector body and at the rearward end against an abutment surface on the plunger 24.

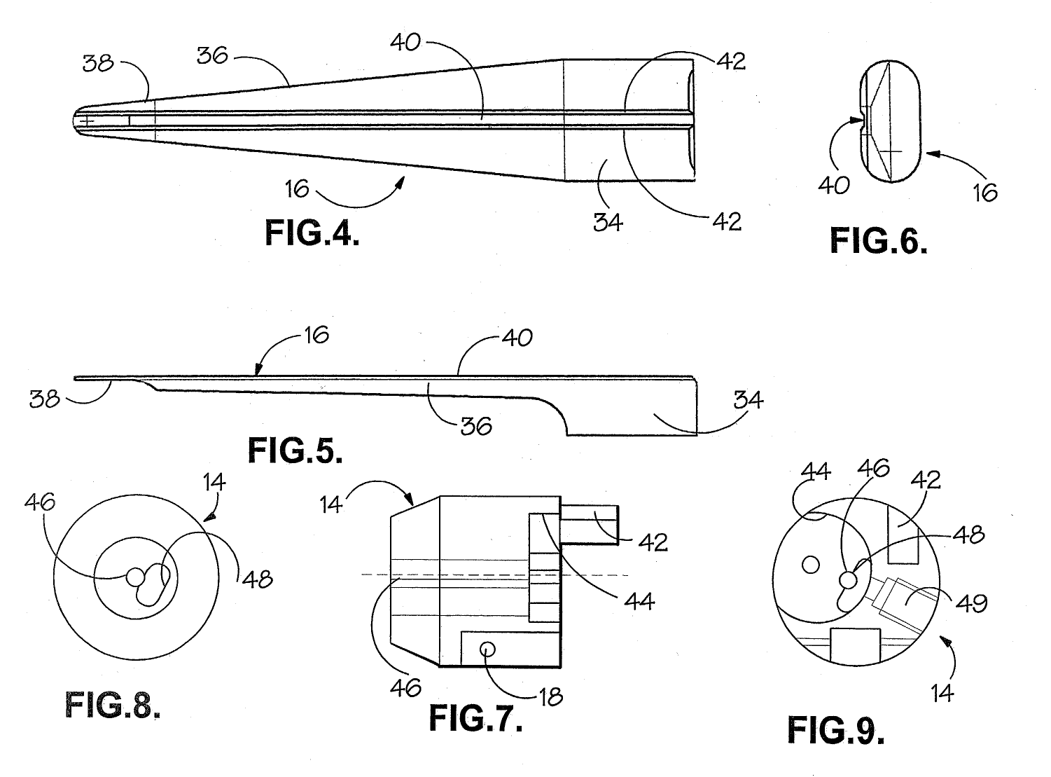

FIGS. 4, 5 and 6 show details of the nose tip 16. In FIG. 3 the nose tip is shown with a curved configuration, whereas in FIG. 5 it is shown as linear. Whether or not the nose tip is provided with a degree of curvature, and the amount of that curvature, is a matter both of choice and of how the instrument is most easily to be used by the surgeon. Different surgical techniques may require nose tips of different configuration. As shown in FIGS. 4 and 5, the nose tip comprises a root portion 34 which is housed within the nose 14, an intermediate portion 36 which tapers both in width and thickness, and a tip portion 38. Along the length of the nose tip 16 there extends a guide channel 40 along which the individual segments to be inserted are pushed. The guide channel 40 has a generally trapezoidal cross-section which is wider at the surface and whose sloping side walls 42 make an angle of 90°.

FIGS. 7 to 9 shown details of the nose 14. The rearward end of the nose 14 includes a locating lug 42 which is received in a slot in the main body 11 of the injector. The nose is pivotable about the pin 18. The rearward face of the nose is provided with a part-circular recess 44 to receive the segment holder 22. It will be appreciated from the location of the recess 44, see FIG. 9 for example, that a portion of the circular segment holder 22 will extend proud of the peripheral surface of the nose 14. Extending through the nose, from the recess 44 to the forward face of the nose, is a central passage 46 through which the segments are arranged to pass in their travel from the holder to the nose tip. The nose 14 is also provided with a generally elliptical aperture 48 which receives the root portion 34 of the nose tip 16. Thus, the dimensions of the aperture 48 match the external dimensions of the nose tip root as shown in FIG. 6. The recess in which the spring, screw and domed pin 23 are located is indicated at 49 in FIG. 9.

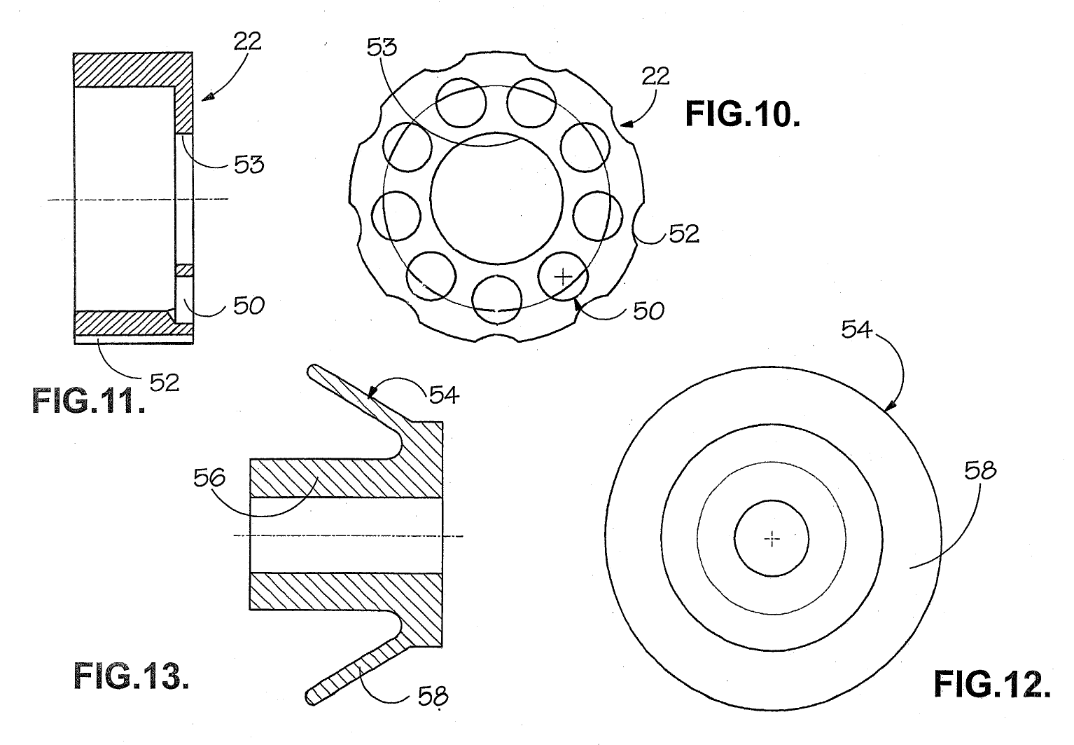

FIGS. 10 and 11 shown the segment holder 22. This is generally circular and is cup-shaped. It is provided through the base of the cup with nine equispaced holes 50 to receive the segments. Around the periphery of the segment holder is provided a series of indentations 52 by means of which the segment holder can be manually indexed on a step-by-step rotational basis. The segment holder has a central hole 53 through its base. The segment holder 22 fits within the recess 44 in the nose 14 and is engaged by the domed pin of the spring-screw-pin combination 23 to provide a ratchet-type indexing facility.

FIGS. 12 and 13 show a segment holder spring 54 which comprises a central cylindrical portion 56 and a circumferential skirt 58 which projects at an angle of some 30° to the longitudinal axis of the central cylindrical portion. The spring 54 is seated within the central aperture 53 through the segment holder 22, with the skirt 58 extending into the chamber defined by the cup-shaped segment holder. This can be seen for example in FIG. 3. The purpose of the spring skirt 58 is to exert pressure against the segments which are loaded in the segment holder 22. By providing this spring pressure one can accommodate different sized segments within the holes 50 in the segment holder.

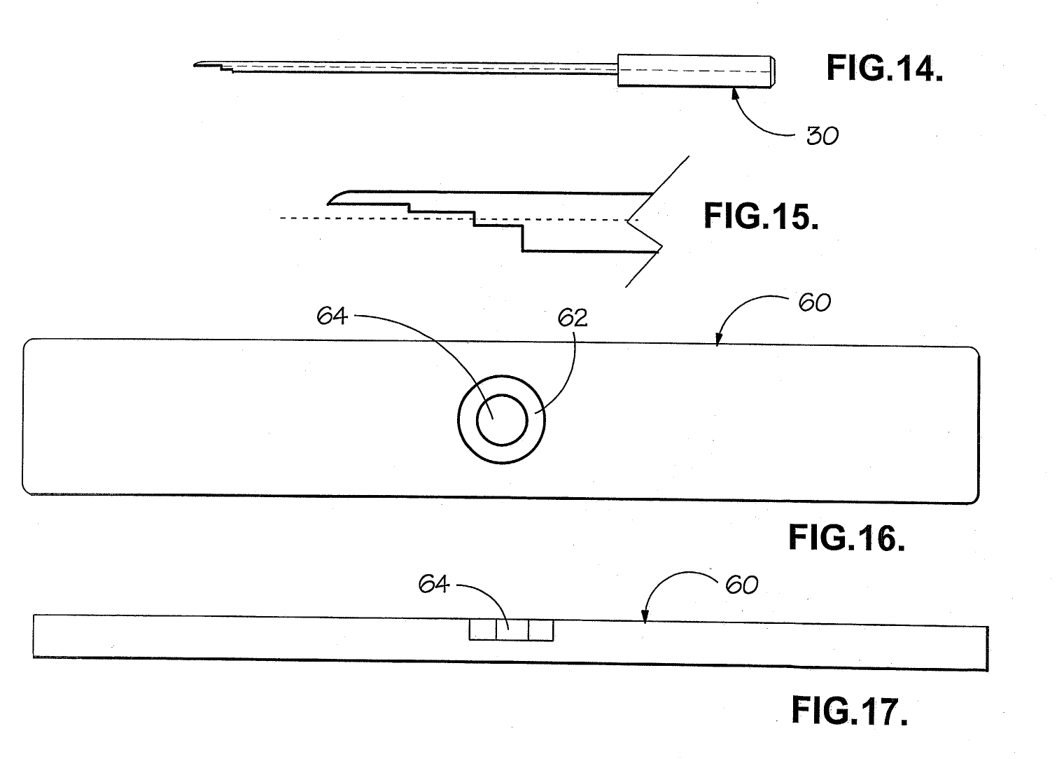

FIGS. 14 and 15 show details of the centre rod 30. The forward end of the centre rod 30, as shown most clearly in

FIG. 15, has a stepped configuration so that the same rod can be used to propel segments of different sizes. All these tip sections of the centre rod will fit in the channel 40 of the nose tip 16.

In use, when the carousel-style segment holder 22 has been loaded with segments of the required dimensions, with the spring 54 exerting pressure against them to hold them in place in the holes 50, the plunger 24 is depressed to advance the centre rod 30 so that its stepped tip engages with the segment in one of the holes 50 and pushes it forward through the nose and through the nose tip, along the guide 40 into the incision in the cornea. When the segment has been inserted the surgeon will then simply rotate the segment holder 22 manually through one step, to align the next segment with the centre rod. The procedure is then repeated.

FIGS. 16 and 17 show a loading plate 60. This is a generally rectangular plate with an annular recess 62 around a spigot 64. The outer diameter of the circular recess 62 is equal to the external diameter of the segment holder 22, and the diameter of the central spigot 64 is equal to the diameter of the hole 53 through the centre of the segment holder. The segment holder 22 can therefore be placed into the loading plate 60 and held there for the insertion first of the segments and then of the segment holder spring 54. The loaded unit can then be transferred to the nose 14.

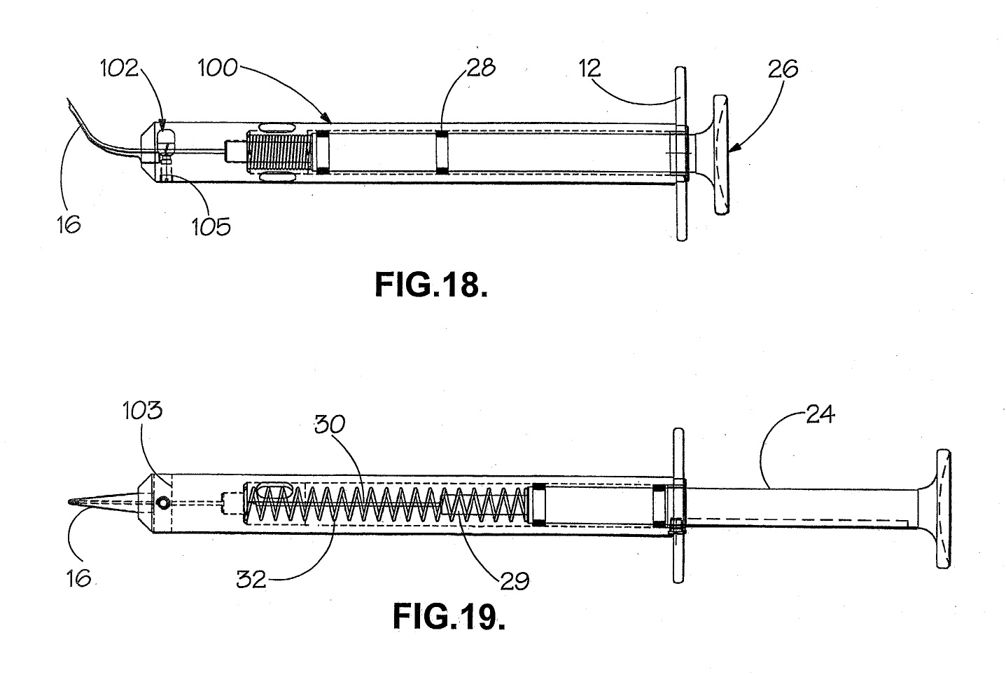

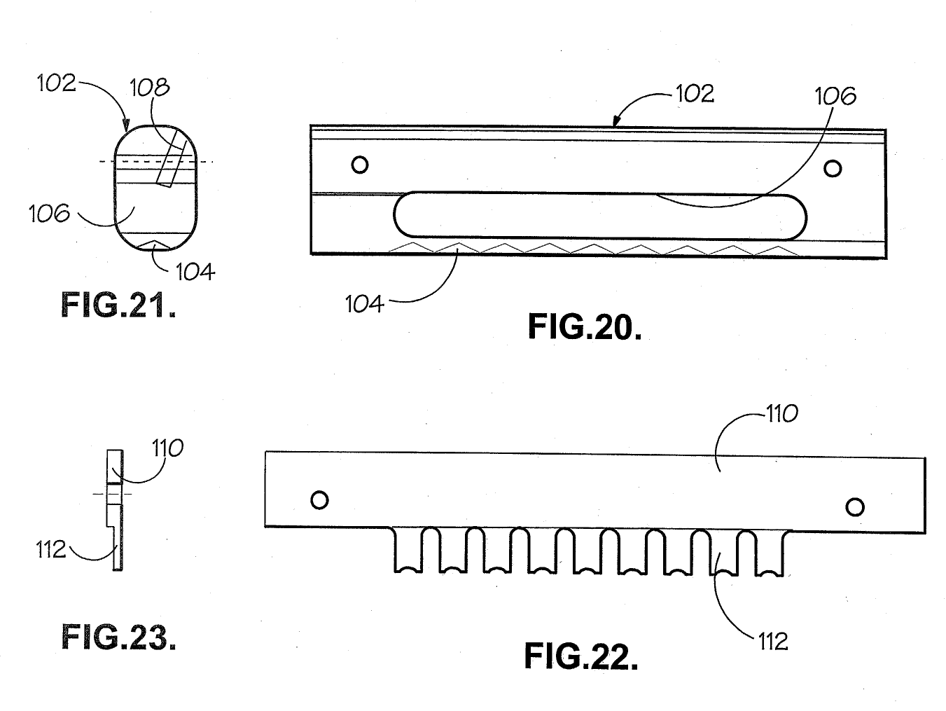

Referring now to FIGS. 18 and 19 there is shown a second embodiment of segment injector in accordance with the invention. Components which are the same as in the first embodiment are indicated by the respective same reference numerals. In this embodiment the injector is not adapted to have the nose portion pivoted open, but instead has a solid barrel 100. The mechanism for dispensing the individual segments is generally the same as in the first embodiment, and the description of those components of the injector will not therefore be described again. In this embodiment, the segment holder is not in the form of a carousel but is in the form of a linear case 102 which is shown most clearly in FIGS. 20 and 21. The case is fitted into the injector so as to extend transversely to the longitudinal axis of the injector. It traverses the barrel 100 through a passageway 103 (FIG. 19) through the barrel. The case is provided with a plurality of indentations 104 along its base by means of which the. case can be advanced linearly in a step-by-step manner. A screw-spring-domed pin combination 105 (FIG. 18) provides a ratchet-type mechanism. The segment holder case 102 is provided with an elongate aperture 106 therethrough. The upper portion of the case 102 is provided with a slot 108 which extends downwards at an angle of some 20° from an upper shoulder of the case down into the through aperture 106. This angled slot 108 extends along substantially the full length of the segment holder case. This angled slot 108 is arranged to receive a segment holder spring 110 which is shown in FIGS. 22 and 23. The length of the segment holder spring 110 is equal to that of the case 102 and is provided with a plurality of nine downwardly extending teeth 112 which are thin and springy. The spring 110 is fitted into the angled slot 108 and is secured to the case by pins towards each end of the spring. The spring teeth 112 extend down into the through aperture 106 and the bottom edge of each spring tooth 112 is shaped with a concave recess so that it will contact and hold a segment between the tooth and the bottom of the aperture 106. Again, because of the springiness of the teeth, segments of different sizes can be accommodated within the same segment holder case. Also, the configuration and shape of the spring teeth can be adapted to the configuration and shape of whatever segment is to be inserted by the injector. The number of spring teeth 112 corresponds to the number of indentations 104 in the bottom of the case, so that the case can be indexed through the injector for the serial insertion of the segments into the incisions in the cornea.

The injectors described above can be modified by being fitted with interchangeable noses and/or nose tips, to enable different segment holders to be used with the same injector.

Also, although the rotatable segment holder has been described as part of a pivotally openable injector, and the linear segment holder as part of an injector with a solid barrel, each type of segment holder can be incorporated in an injector of the other type.

1. An ophthalmic injector comprising a barrel, a plunger displaceable longitudinally within the barrel, a nose tip forwardly of the barrel, and a segment holder arranged to hold a plurality of segments to be inserted into incisions in the eye, the segment holder being displaceably stepwise and being positioned so that the segments are engageable in turn by the plunger or an extension thereof to push them from the holder through the nose tip.

2. An ophthalmic injector according to claim 1, in which the segment holder projects out beyond the contour of the barrel to permit manual stepwise movement of the segment holder.

3. An ophthalmic injector according to claim 1 or 2, in which the segment holder has associated means to hold the segments in the holder under spring bias until pushed from the holder.

4. An ophthalmic injector according to claim 1, 2 or 3, in which the segment holder is a rotatable carousel-type holder with the segments arranged to be held on the locus of a circle.

5. An ophthalmic injector according to claim 4, in which the rotatable segment holder is positioned offset from the longitudinal axis of the barrel and plunger so that rotation of the holder brings the segments in turn into alignment with said longitudinal axis for dispensation.

6. An ophthalmic injector according to claim 5, in which the holder has peripheral surface indentations by means of which it can be manually indexed.

7. An ophthalmic injector according to claim 4, 5 or 6, in which the segment holder is cup-shaped with the base of the cup having equispaced holes to receive the segments.

8. An ophthalmic injector according to claim 7, in which a spring is mounted within the cup-shaped holder, the spring having a central cylindrical portion and an inclined circumferential skirt which projects at an angle of about 30° to the longitudinal axis of the injector and bears against the segments.

9. An ophthalmic injector according to claim 1, 2 or 3, in which the segment holder is linearly movable transversely of the longitudinal axis of the barrel and plunger.

10. An ophthalmic injector according to claim 9, in which the linear segment holder is indexable by a ratchet mechanism.

11. An ophthalmic injector according to claim 9 or 10, in which the segment holder comprises a case provided with a plurality of indentations by means of which it can be indexed through the barrel.

12. An ophthalmic injector according to claim 9, 10 or 11, in which the segment holder is provided with a slot extending substantially the full length of the holder, the slot being angled downwards at an angle of about 20° from an upper shoulder of the holder, with an elongate spring located within said slot, the spring having a plurality of downwardly extending teeth equal in number to the number of segments which can be carried by the holder and engageable respectively with segments located in the holder.

13. An ophthalmic injector according to any preceding claim, in which a nose is mounted at the forward end of the barrel, and the nose tip is mounted at the forward end of the nose, the nose and nose tip being pivotable relative to the barrel through 90°.

14. An ophthalmic injector according to any preceding claim, in which the nose tip has a guide channel for the segments extending the length of the tip.

15. An ophthalmic injector according to claim 14, in which the guide channel is of trapezoidal cross-section.

16. An ophthalmic injector according to any preceding claim, in which the forward end of the plunger has a stepped configuration.

17. An ophthalmic injector according to any preceding claim, in which the segment holder is arranged to hold nine segments.

18. An ophthalmic injector according to any preceding claim, in which the segment holder is adapted to hold segments suitable for the treatment of hyperopia.
